(19) United States
(12) Patent Application Publication
(10) Pub. No.: US 2023/0380778 A1
(43) Pub. Date: Nov. 30, 2023

(54) METHODS, SYSTEMS, DEVICES, AND STORAGE MEDIA FOR TRACER CLASSIFICATION

(71) Applicant: SHANGHAI UNITED IMAGING HEALTHCARE CO., LTD., Shanghai (CN)

(72) Inventor: Huifang XIE, Shanghai (CN)

(73) Assignee: SHANGHAI UNITED IMAGING HEALTHCARE CO., LTD., Shanghai (CN)

(21) Appl. No.: 18/325,095

(22) Filed: May 29, 2023

(30) Foreign Application Priority Data
May 30, 2022 (CN) 202210602642.3

(51) Int. Cl. A61B 6/03; G06T 5/00; G06V 10/25; G06V 10/44; G06V 10/764; G06V 10/771

(52) U.S. Cl. CPC A61B 6/037; G06T 5/002; G06V 10/25; G06V 10/44; G06V 10/764; G06V 10/771; G06T 2207/10088

(57) ABSTRACT

The embodiments of the present disclosure provide a method for classifying a tracer, a system and a device, and a storage medium. The method for classifying the tracer comprises obtaining imaging data related to an emission computed tomography (ECT) scan of a target object, the target object being injected with a tracer during the ECT scan; and determining classification information of the tracer by processing the imaging data using a tracer classification model, the tracer classification model being a trained machine learning model.

### CROSS-REFERENCE TO RELATED APPLICATIONS

This application claims priority to Chinese Patent Application No. 202210602642.3, filed on May 30, 2022, the entire contents of which are incorporated herein by reference.

### TECHNICAL FIELD

The present disclosure relates to the field of medical scanning, and in particular to methods, systems, devices, and storage media for tracer classification.

### BACKGROUND

In an emission computed tomography scan (e.g., a positron emission computed tomography (PET) scan, a single photon emission computed tomography (SPECT) scan, etc.), the radionuclide, drug, or tracer injected into the scanned subject will directly affect the quality of the resulting image. Moreover, image reconstruction based on scan data collected using a specific tracer will involve adjustments to the reconstruction algorithm based on the used tracer. In order to further ensure the effective use of the scan data and improve the quality of image reconstruction, the type of the used tracer needs to be determined before image reconstruction. Therefore, it is desirable to provide methods and systems for classifying a tracer used in an ECT scan based on scan data.

### SUMMARY

In order to quickly, automatically, and accurately determine classification information of a tracer used in an ECT scan by processing imaging data collected via the ECT scan, and improve the accuracy of subsequent processing (e.g., image reconstruction, etc.) on the imaging data based on the classification information of the tracer, one of the embodiments of the present disclosure provides a method for classifying a tracer. The method for classifying the tracer may comprise: obtaining imaging data related to an ECT scan of a target object, the target object being injected with a tracer during the ECT scan; and determining classification information of the tracer by processing the imaging data using a tracer classification model, the tracer classification model being a trained machine learning model.

One of the embodiments of the present disclosure provides a system. The system comprises at least one storage device storing a set of instructions for classifying a tracer, and at least one processor configured to communicate with the at least one storage device. When executing the set of instructions, the at least one processor is configured to direct the system to perform operations including: obtaining imaging data related to an emission computed tomography (ECT) scan of a target object, the target object being injected with a tracer during the ECT scan; and determining classification information of the tracer by processing the imaging data using a tracer classification model, the tracer classification model being a trained machine learning model.

One of the embodiments of the present disclosure provides a non-transitory computer readable medium, which comprises a set of instructions for classifying a tracer. When executed by at least one processor, the set of instructions direct the at least one processor to effectuate a method. The method comprising: obtaining imaging data related to an emission computed tomography (ECT) scan of a target object, the target object being injected with a tracer during the ECT scan; and determining classification information of the tracer by processing the imaging data using a tracer classification model, the tracer classification model being a trained machine learning model.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further illustrated in terms of exemplary embodiments. These exemplary embodiments are described in detail with reference to the drawings. These embodiments are non-limiting. In these embodiments, the same number indicates the same structure, wherein:

FIG. 1 is a schematic diagram illustrating an exemplary tracer classification system according to some embodiments of the present disclosure;

FIG. 2 is a block diagram illustrating an exemplary tracer classification system according to some embodiments of the present disclosure;

FIG. 3 is a flowchart illustrating an exemplary process for classifying a tracer according to some embodiments of the present disclosure;

FIG. 4A is a schematic diagram illustrating training a tracer classification model according to some embodiments of the present disclosure;

FIG. 4B is a schematic diagram illustrating training a tracer classification model according to some embodiments of the present disclosure;

FIG. 5 is a flowchart illustrating an exemplary process for determining classification information of a tracer according to some embodiments of the present disclosure;

FIG. 6 is a flowchart illustrating an exemplary process for determining classification information of a tracer according to some embodiments of the present disclosure;

FIG. 7 is a flowchart illustrating an exemplary process for determining classification information of a tracer according to some embodiments of the present disclosure;

FIG. 8 is a flowchart illustrating an exemplary process for determining classification information of a tracer according to some embodiments of the present disclosure;

FIG. 9 is a flowchart illustrating an exemplary process for determining classification information of a tracer according to some embodiments of the present disclosure; and

FIG. 10 is a flowchart illustrating an exemplary process for determining classification information of a tracer according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “device,” “unit,” “module,” and/or “block” used herein are one method to distinguish different components, elements, parts, sections or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may apply to a system, a device, or a portion thereof.

It will be understood that when a unit, device, module or block is referred to as being “on,” “connected to,” or “coupled to,” another unit, device, module, or block, it may be directly on, connected or coupled to, or communicate with the other unit, device, module, or block, or an intervening unit, device, module, or block may be present, unless the context clearly indicates otherwise. As used herein, the term “and/or” includes any and all combinations of one or more of the associated listed items.

The term “pixel” and “voxel” in the present disclosure are used interchangeably to refer to an element in an image. An anatomical structure shown in an image of an object (e.g., a patient) may correspond to an actual anatomical structure existing in or on the object's body. For example, a body part shown in an image may correspond to an actual body part existing in or on the object's body, and a feature point in an image may correspond to an actual physical point existing in or on the object's body. For the convenience of descriptions, an anatomical structure shown in an image and its corresponding actual anatomical structure are used interchangeably. For example, the chest of the object refers to the actual chest of the object or a region representing the chest in an image of the object. The term “image” in the present disclosure is used to refer to images of various forms, including a 2-dimensional image, a 3-dimensional image, a 4-dimensional image, etc.

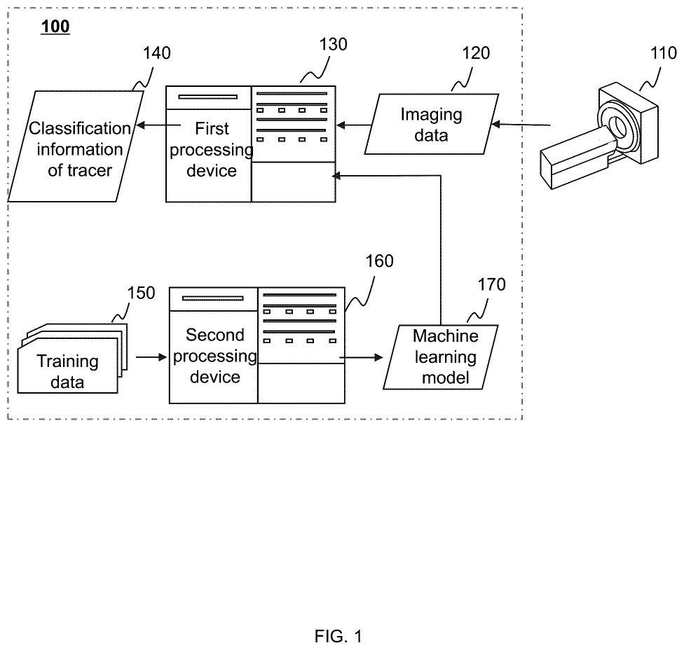

FIG. 1 is a schematic diagram illustrating an exemplary tracer classification system 100 according to some embodiments of the present disclosure.

In an ECT scan, sometimes a target object may need to be injected with a tracer, such as 18F-fluorodeoxyglucose (FDG), radiolabeled amino acid (e.g., 18F amino acid tracer FET), choline derivative (e.g., gallium 68 or 18F prostate-specific membrane antigen PSMA), etc. In some embodiments, the target object may be injected with a plurality of tracers simultaneously. The tracer classification system 100 may determine classification information of the tracer used in the ECT scan based on imaging data obtained in the ECT scan. If the target object is injected with the plurality of tracers simultaneously, the tracer classification system 100 may determine type information of each tracer. In some embodiments, the tracer classification system 100 may train a machine learning model 170 (e.g., a tracer classification model and/or an enhancement model), and use the machine learning model in determining the classification information of the tracer.

For example, as shown in FIG. 1, the tracer classification system 100 may include a first processing device 130 and a second processing device 160.

The first processing device 130 may obtain imaging data 120 related to the target object, and determine classification information of a tracer used when the imaging data 120 is collected by processing the imaging data 120. The target object may include the whole or a part of a human or animal injected with a tracer, such as the heart.

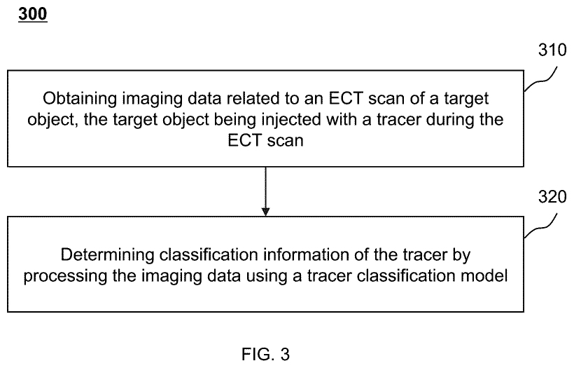

The imaging data 120 may include raw data collected by an ECT imaging device 110 during a scan of the target object, image data reconstructed based on the raw data, intermediate data generated during the process of generating the image data, etc., or any combination thereof. More descriptions about the imaging data 120 may be found in FIG. 3, which is not repeated here.

Exemplary ECT imaging devices 110 may include a positron emission tomography (PET) scanner, a positron emission tomography/computed tomography (PET-CT) scanner, a positron emission tomography/magnetic resonance imaging (PET-MRI) scanner, a single photon emission computed tomography (SPECT) scanner, etc., or a combination thereof.

In some embodiments, the first processing device 130 may determine classification information 140 of the tracer based on a machine learning model 170. The machine learning model 170 may be generated by the second processing device 160 based on training data 150. For example, the second processing device 160 may generate a tracer classification model through model training based on reference imaging data with known tracer classification information. The model training may be performed based on one or more machine learning algorithms, such as an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, rule-based machine learning algorithm, etc., or any combination thereof. More descriptions about the tracer classification model and the classification information 140 of the tracer may be found in FIGS. 3-10, which is not repeated here.

The first processing device 130 and the second processing device 160 may refer to a system with computing capability, which may include various computers, such as a server and a personal computer, or a computing platform composed of a plurality of computers connected in various manners.

The first processing device 130 and the second processing device 160 may include a processor. The processor may execute program instructions. The processor may include one or more of a universal central processing unit (CPU), a graphics processing unit (GPU), a microprocessor unit (MPU), an application-specific integrated circuit (ASIC), or other types of integrated circuits.

In some embodiments, the first processing device 130 and the second processing device 160 may include a storage device. The storage device may store instructions or data. The storage device may include a mass memory, a removable memory, a volatile read-write memory, a read-only memory (ROM), etc., or any combination thereof.

In some embodiments, the first processing device 130 and the second processing device 160 may further include a communication port for internal connection and external connection and/or a terminal for input or output. The communication port may be connected to a wired network and/or a wireless network. The terminal may include various devices with functions of receiving and/or sending information, such as a computer, a mobile phone, a text scanning device, a display device, a printer, etc.

It should be noted that the above description about the tracer classification system 100 is provided for illustrative purposes only, and is not intended to limit the scope of the embodiments of the present disclosure. Those skilled in the art may make various modifications or alterations based on the descriptions of the embodiments of the present disclosure. For example, the first processing device 130 and the second processing device 160 may be implemented by the same processing device. As another example, the second processing device 160 may not be arranged in the tracer classification system 100, and the first processing device 130 may obtain a pre-trained machine learning model 170 from an external device or its own storage device. As another example, the tracer classification system 100 may further include a storage device for storing data and/or instructions. However, these alterations and modifications do not depart from the scope of the embodiments of the present disclosure.

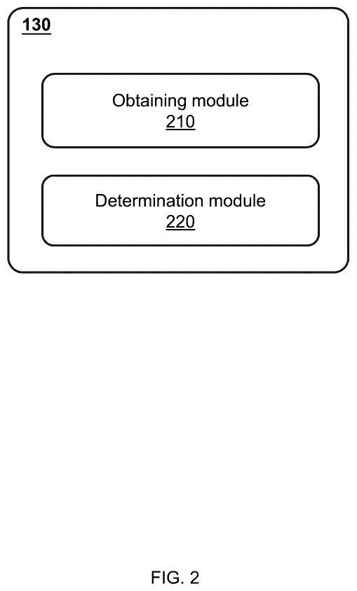

FIG. 2 is a schematic diagram illustrating an exemplary tracer classification system 200 according to some embodiments of the present disclosure. As shown in FIG. 2, the tracer classification system 200 may include an obtaining module 210 and a determination module 220. In some embodiments, the tracer classification system 200 may be implemented by the first processing device 130 described in FIG. 1. In other words, the first processing device 130 may include the obtaining module 210 and the determination module 220.

In some embodiments, the obtaining module 210 may be configured to obtain imaging data related to an ECT scan of a target object injected with a tracer. The imaging data may include raw data obtained by an ECT imaging device in the ECT scan, or image data generated after processing the raw data. Merely by way of example, the imaging data may include PET scan data, list-mode raw data, a PET image, and the like. The tracer may be a marker injected into the target object. More descriptions about the imaging data and the tracer may be found in operation 310 in FIG. 3.

In some embodiments, the determination module 220 may determine classification information of the tracer by processing the imaging data using a tracer classification model, the tracer classification model being a trained machine learning model. More descriptions about the tracer classification model may be found in other parts of the present disclosure, which is not repeated here.

In some embodiments, the determination module 220 may be further configured to determine at least one feature of the imaging data; and determine the classification information of the tracer by processing the imaging data and the at least one feature using the tracer classification model. In some embodiments, the at least one feature of the imaging data may be related to the type of the imaging data, and different types of imaging data may correspond to the same feature or different features. More descriptions about the at least one feature of the imaging data may be found in operation 320 in FIG. 3.

In some embodiments, the determination module 220 may be further configured to determine the classification information of the tracer by processing the imaging data using the tracer classification model and an enhancement model. In some embodiments, the determination module 220 may determine the classification information of the tracer by processing the imaging data based on the tracer classification model and the enhancement model through one or more iterations.

In some embodiments, an iteration performed by the determination module 220 may include: determining initial classification information of the tracer by processing initial imaging data of the iteration using the tracer classification model; generating updated imaging data by performing noise reduction processing and/or detail enhancement processing on the initial imaging data using the enhancement model; determining whether an iteration termination condition is satisfied; and designating the initial classification information as the classification information of the tracer in response to a determination result that the iteration termination condition is satisfied; or designating the updated imaging data as initial imaging data of a next iteration in response to a determination result that the iteration termination condition is not satisfied.

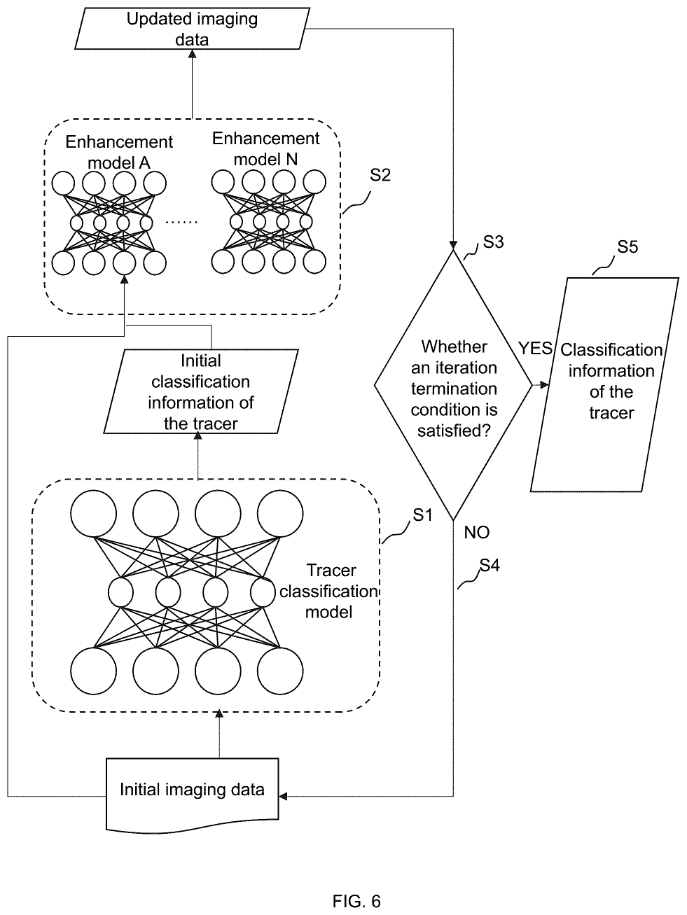

In some embodiments, the determination module 220 may generate updated imaging data by performing noise reduction processing on the initial imaging data using the enhancement model by: obtaining at least two candidate enhancement models corresponding to at least two tracer types; selecting the enhancement model from the at least two candidate enhancement models based on the initial classification information; and generating the updated imaging data by performing the noise reduction processing and/or detail enhancement processing on the initial imaging data using the enhancement model. More descriptions about the iterative operation and performing the noise reduction processing on the initial imaging data by selecting the enhancement model based on the tracer type may be found in FIG. 6 and related descriptions thereof.

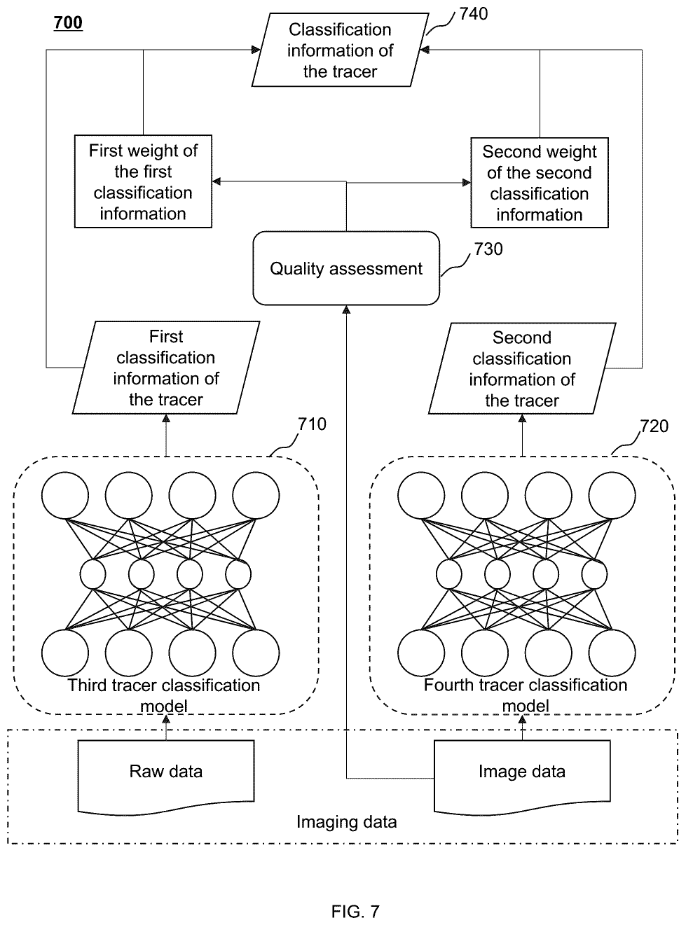

In some embodiments, the imaging data may include raw data and image data collected based on the ECT scan of the target object, and the determination module 220 may determine the classification information of the tracer by: determining first classification information of the tracer by processing the raw data using a tracer classification model corresponding to the raw data; determining second classification information of the tracer by processing the image data using the tracer classification model corresponding to the image data; determining a first weight of the first classification information and a second weight of the second classification information by performing quality assessment on the image data; and determining the classification information of the tracer based on the first classification information, the second classification information, the first weight, and the second weight. More descriptions about the first classification information, the second classification information, the first weight, and the second weight may be found in the corresponding descriptions of FIG. 7.

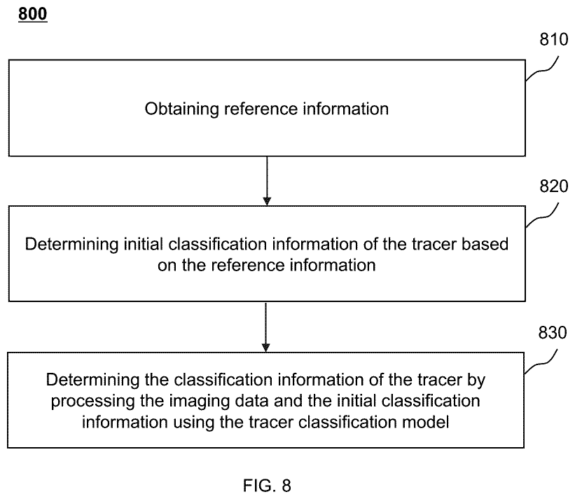

In some embodiments, the determination module 220 may determine the classification information of the tracer by: obtaining reference information; determining initial classification information of the tracer based on the reference information; and determining the classification information of the tracer by processing the imaging data and the initial classification information using the tracer classification model. More descriptions about the reference information and the initial classification information may be found in the corresponding descriptions of FIG. 8.

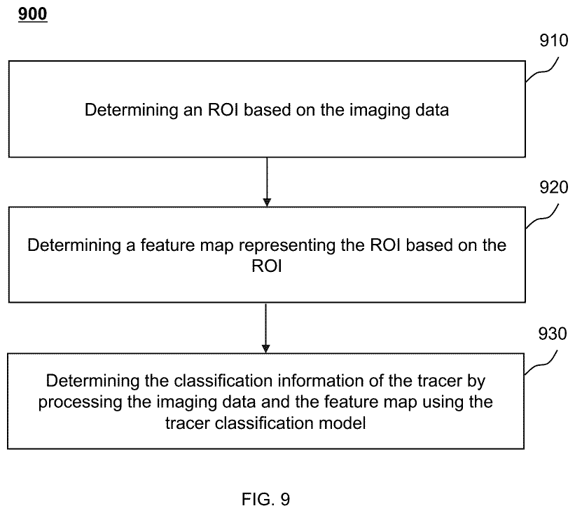

In some embodiments, the determination module 220 may determine classification information of the tracer by: determining a region of interest based on the imaging data; determining a feature map representing the region of interest based on the region of interest; and determining the classification information of the tracer by processing the imaging data and the feature map using the tracer classification model. More descriptions about the region of interest and the feature map may be found in the corresponding descriptions of FIG. 9.

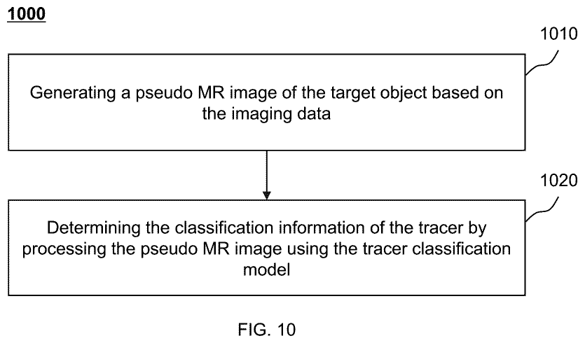

In some embodiments, the determination module 220 may determine classification information of the tracer by: generating a pseudo magnetic resonance (MR) image of the target object based on the imaging data; and determining the classification information of the tracer by processing the pseudo MR image using the tracer classification model. More descriptions about the pseudo MR image may be found in the corresponding descriptions of FIG. 10.

It should be understood that the tracer classification system 200 and modules thereof may be implemented in various ways. For example, in some embodiments, the system and modules thereof may be implemented by hardware, software, or a combination of software and hardware. The hardware may be implemented using a dedicated logic; and the software may be stored in memory and executed by a suitable instruction execution system, such as a microprocessor or specially designed hardware. Those skilled in the art would appreciate that the methods and systems described above may be implemented using computer-executable instructions and/or control codes contained in a processor, for example, a carrier medium such as a magnetic disk, CD, or DVD-ROM, a programmable memory such as a read-only memory (firmware), or a data carrier such as an optical or electronic signal carrier. The system and modules thereof of the present disclosure may not only be implemented by a hardware circuit such as a very large scale integrated circuit or gate array, a semiconductor such as a logic chip, a transistor, etc., or a programmable hardware device such as a field programmable gate array, a programmable logic device, etc., may be implemented by software executed by various types of processors, or may be implemented by a combination (e.g., firmware) of the above hardware circuits and software.

It should be noted that the above description of the tracer classification system 200 is merely provided for the purposes of illustration, and not intended to limit the scope of the present disclosure. It can be understood that for those skilled in the art, after understanding the principle of the system, various modules may be combined arbitrarily, or a subsystem may be formed to connect with other modules without departing from the principle. Such variations are all within the protection scope of the embodiments of the present disclosure.

For example, the obtaining module 210 and the determination module 220 may share one storage module, or each module may have its own storage module. For another example, the tracer classification system 200 may further include a training module for generating one or more machine learning models (e.g., an enhancement model, a tracer classification model, a quality assessment model, etc.) disclosed in the present disclosure. In some embodiments, the training module and other modules described above may be implemented on different computing devices. Merely by way of example, the training module may be implemented on a computing device of a vendor of the machine learning model(s) used for tracer classification (e.g., the second processing device 160), while the other modules described above may be implemented on a computing device of a user of the machine learning model(s) (e.g., the first processing device 130).

FIG. 3 is a flowchart illustrating an exemplary process 300 for classifying a tracer according to some embodiments of the present disclosure.

As shown in FIG. 3, the process 300 may include the following operations.

In 310, imaging data related to an ECT scan of a target object may be obtained, the target object being injected with a tracer during the ECT scan. In some embodiments, operation 310 may be performed by the obtaining module 210.

In some embodiments, the imaging data may include original data (i.e., raw data) obtained by an ECT imaging device when scanning the target object, such as PET scan data, list-mode raw data, sinogram data, etc. In some embodiments, the imaging data may include image data generated after processing the raw data, such as a PET image, time-of-flight (TOF-histo) image data, an activity distribution image, etc. In some embodiments, the imaging data may include data or images relating to an intermediate result generated in the process of generating the image data based on the raw data.

In some embodiments, the imaging data may include imaging data related to one or more ECT scans of the target object. For example, the imaging data may be PET scan data collected during a certain PET scan of the target object. As another example, the imaging data may include images collected in a plurality of PET scans of the target object. The same or different tracers may be injected in the plurality of PET scans.

In some embodiments, the obtaining module 210 may obtain imaging data related to a plurality of ECT scans of a plurality of target objects. For example, the imaging data may include PET scan data obtained in a plurality of PET scans of the plurality of target objects. The same or different tracers may be injected in the plurality of scans of the plurality of target objects.

The imaging data may be obtained in various ways. In some embodiments, the imaging data may be obtained directly from the ECT imaging device or a storage device. In some embodiments, the imaging data may be obtained by processing the raw data obtained by the ECT imaging device. The processing of the raw data may include format conversion, data filtering, image reconstruction, etc. For example, an activity distribution image may be obtained after performing image reconstruction on the raw data, and the activity distribution image may be used as the imaging data.

The target object may be a human body or an animal injected with the tracer. The tracer may be injected before or during the scan. Alternatively, one type of tracer may be injected before a first scan phase, another type of tracer may be injected after the first scan phase, and after a preset time, a second scan phase may be performed on the target object. In some embodiments, the target object may be injected with a plurality of tracers simultaneously.

In some embodiments, the target object may be the whole human body or animal, or part of the human body or animal (e.g., a specific organ or tissue of a patient). The organ may include, but be not limited to, a brain, a lung, a heart, a kidney, a liver, and the like. The tissue may include, but be not limited to, epithelial tissue, connective tissue, nerve tissue, muscle tissue, and the like. The tracer may be a marker injected into the target object for observing, studying, and measuring behaviors or properties of a substance during a given process. In some embodiments, the tracer may include a radionuclide and an organic ligand to which the radionuclide is attached. For example, common radionuclides may include F-18, Ga-68, N-13, C-11, etc. Common organic ligands may include FDG, amino acids, choline, proteins, etc.

In 320, classification information of the tracer may be determined by processing the imaging data using a tracer classification model. In some embodiments, operation 320 may be performed by the determination module 220.

The classification information of the tracer may refer to classification information of the tracer used when the imaging data of the target object is collected. For example, the classification information of the tracer may include a tracer type, a probability of belonging to a certain tracer type, and the like. In some embodiments, the tracer may be classified based on the type of the radionuclide and/or the type of the organic ligand. Merely by way of example, based on the radionuclide, the tracer may be classified into an F-18 tracer, a Ga-68 tracer, a C-11 tracer, an N-13 tracer, an 0-14 tracer, an 1-124 tracer, an Rb-82 tracer, a Ge-68 tracer, a Na-22 tracer, a Cu-62 tracer, an Mn-52 tracer, a Br-75 Tracer, a K-38 tracer, a Y-86 tracer, a Zr-89 tracer, a Cu-64 tracer, a Y-90 tracer, an 0-15 tracer, a Br-76 tracer, etc. In some embodiments, each radionuclide may be classified into one type of tracer. Alternatively, a plurality of radionuclides may be combined as a broad type of tracers. As another example, based on the organic ligand, the tracer may be classified into an FDG tracer, an amino acid tracer, a choline tracer, a protein tracer, an FLT tracer, an FMISO tracer, an FMISO tracer, an Amyvid tracer, a NaF tracer, etc. As another example, based on the nuclide and the organic ligand together, a specific type (e.g., 11C-MET, 18F-FDG, 18F-FLT, etc.) of the tracer may be determined.

The tracer classification model may refer to a machine learning model that can output the classification information of the tracer by processing the imaging data. For example, the tracer classification model may include a convolutional neural network model (CNN), a Naive Bayesian classification model, a Gaussian distribution Bayesian classification model, a KNN classification model, etc., or any combination thereof. In some embodiments, the target object may be injected with a plurality of tracers simultaneously. The tracer classification model may determine the type information of each tracer.

In some embodiments, the determination module 220 may determine an input of the tracer classification model based on the imaging data, and feed the input into the tracer classification model. The tracer classification model may output the corresponding classification information of the tracer.

In some embodiments, the input of the tracer classification model may include the imaging data or processed imaging data. For example, the determination module 220 may perform preprocessing (e.g., noise reduction) on the imaging data, and use the preprocessed imaging data as the input of the tracer classification model. In some embodiments, the determination module 220 may perform noise reduction processing and/or detail enhancement processing on the imaging data using an enhancement model, and use the imaging data after noise reduction as the input of the tracer classification model.

In some embodiments, the preprocessing of the imaging data may further include a data feature enhancement operation (e.g., edge information enhancement), a data normalization operation (e.g., normalizing all images to a similar event count level or a similar noise level), a data correction operation (e.g., scattering correction, random correction, attenuation correction), etc. In some embodiments, the preprocessing may further include format adjustment (e.g., image parameter adjustment) to generate clearer imaging data. More descriptions about the adjustment of the imaging data may be found in FIG. 10 and relevant descriptions thereof.

In some embodiments, the imaging data may include both the raw data and the image data collected based on the ECT scan of the target object. The determination module 220 may determine the classification information of the tracer by processing the raw data and the image data using the tracer classification model. More descriptions about determining the classification information of the tracer based on the raw data and the image data may be found in the relevant descriptions of FIG. 7.

In some embodiments, the determination module 220 may obtain reference information, and determine initial classification information of the tracer based on the reference information. The determination module 220 may further determine the classification information of the tracer by inputting the imaging data and the initial classification information into the tracer classification model. More descriptions about determining the classification information of the tracer based on the imaging data and the initial classification information may be found in relevant descriptions of FIG. 8.

In some embodiments, the determination module 220 may determine a region of interest based on the imaging data, and then determine a feature map representing the region of interest. Further, the determination module 220 may determine the classification information of the tracer by inputting the imaging data and the feature map into the tracer classification model. More descriptions about determining the classification information of the tracer based on the imaging data and the feature map may be found in relevant descriptions of FIG. 9.

In some embodiments, the determination module 220 may generate a pseudo MR image of the target object based on the imaging data. Further, the determination module 220 may determine the classification information of the tracer by inputting the pseudo MR image into the tracer classification model. More descriptions about determining the classification information of the tracer based on the pseudo MR image may be found in relevant descriptions of FIG. 10.

In some embodiments, the determination module 220 may determine at least one feature of the imaging data, and use the at least one feature and the imaging data as the input of the tracer classification model. The tracer classification model may output the classification information of the tracer by processing the imaging data and the at least one feature.

In some embodiments, the types of extracted data features for different types of imaging data may be the same or different. For example, the type of features extracted from image data may be different from the type of features extracted from raw data.

Merely by way of example, when the imaging data is a brain image, the at least one feature of the imaging data may include feature(s) of a region of interest (ROI), or feature(s) of the ROI relative to a reference region. Merely by way of example, one or more of the following information may be used as the at least one feature of the imaging data: an activity ratio of a basal ganglia to other regions, a shape of the basal ganglia, a size of the basal ganglia, an activity value and an activity distribution pattern of a gray matter region, an activity value and an activity distribution pattern of a white matter area, an activity ratio of the gray matter region to the white matter region, an activity of a scalp region, an activity of a cerebrospinal fluid region, an image resolution, etc.

In some embodiments, if the imaging data includes images corresponding to different times, a changing trend of certain features in the images over time (e.g., a change in the overall gradient of multiple images) may be used as the at least one feature of the imaging data.

In some embodiments, if the imaging data includes PET raw data, the at least one feature of the imaging data may include count information of one or more types of events in the PET scan, a file size of the raw data, etc. The count information may include total count information and/or count rate information. The total count information may include a coincidence event count, a random event count, a scattering event count, a noise equivalent count, etc. The count rate information may include, for example, a coincidence event count rate, a random event count rate, a scattering event count rate, a noise equivalent count rate, etc.

In some embodiments, the classification accuracy and efficiency of the tracer may be improved by performing feature extraction on the imaging data, and then further processing the imaging data and the extracted feature data using the tracer classification model.

In some embodiments, the imaging data obtained with different tracers may typically have different features. For example, for a tracer that has a high uptake in a certain region of the human body, pixel values of a corresponding sinogram at different angles corresponding to the region may be relatively high (e.g., exceeding a preset pixel threshold), and pixel values in other regions may be relatively low. However, for a tracer that has a uniform uptake in the human body, a sinogram corresponding to the tracer, pixel values of a corresponding sinogram may be evenly distributed. Since imaging data collected by using different tracers have different features, taking the data features as part of the input of the tracer classification model may provide more classification bases for the tracer classification model, thereby improving the accuracy of the classification result. Besides, the amount of calculation for feature extraction of the tracer classification model can be reduced and the time required for classification can be shortened.

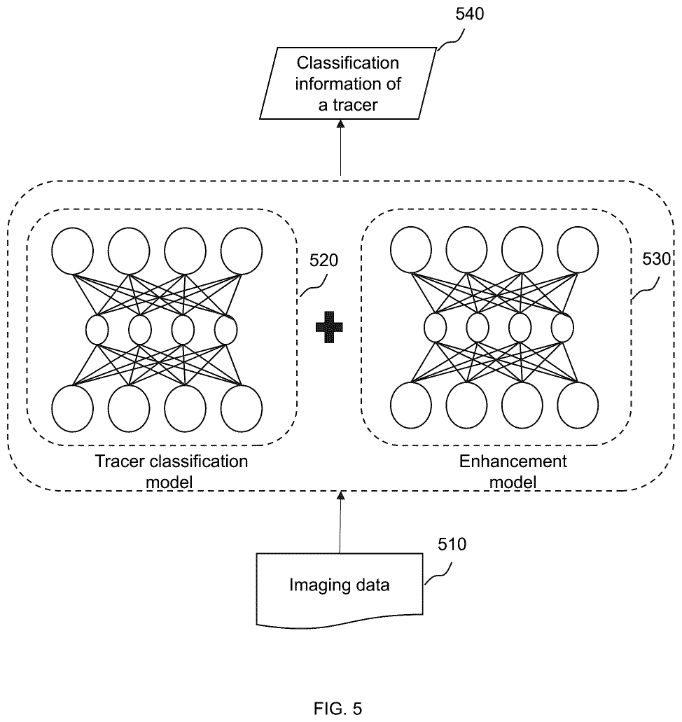

In some embodiments, the determination module 220 may determine the classification information of the tracer by processing the imaging data using the tracer classification model and an enhancement model. More descriptions about determining the classification information of the tracer based on the tracer classification model and the enhancement model may be found in relevant descriptions of FIG. 5 and FIG. 6, which is not repeated here.

In some embodiments, the tracer classification model may be a first tracer classification model or a second tracer classification model. An input of the first tracer classification model may include imaging data corresponding to a single ECT scan, and an output of the first tracer classification model may include classification information of the tracer used in the ECT scan. An input of the second tracer classification model may include imaging data corresponding to a plurality of ECT scans of one or more target objects, and an output of the second tracer classification model may include classified imaging data based on the tracer type. For example, the second tracer classification model may divide a plurality of PET images collected by a plurality of PET scans into several image sets based on the corresponding tracer types. Each image set may correspond to one specific tracer type. In some embodiments, the target object may be injected with the plurality of tracers simultaneously. The first tracer classification model or the second tracer classification model may determine the type information of each tracer.

In some embodiments, the obtained classification information of the tracer may have further applications, e.g., scan protocol adjustment and/or reconstruction parameter adjustment. For example, a scanning protocol may be adjusted based on the classification information to generate a target scan protocol. As another example, one or more reconstruction parameters used in the reconstruction of the imaging data may be determined based on the classification information.

Merely by way of example, image reconstruction may be performed on the imaging data based on the classification information of the tracer obtained by the tracer classification model. For example, for the imaging data corresponding to a specific type of tracer, the image reconstruction may be performed using a reconstruction algorithm and reconstruction parameters corresponding to the type of tracer. That is to say, the reconstruction algorithm and the reconstruction parameters used for image reconstruction of the imaging data may be determined based on the type of tracer used in the collection of the imaging data. In some embodiments, the reconstruction algorithms and the reconstruction parameters corresponding to each type of tracer may be predetermined. Therefore, the accuracy and efficiency of image reconstruction can be improved.

In some embodiments, the region of interest may be determined based on the classification information of the tracer. For example, an image may be reconstructed based on imaging data collected using a prostate specific membrane antigen (PSMA) tracer, and a prostate region may be marked in the image; or a target part corresponding to the prostate region may be determined from the imaging data, and the image corresponding to the prostate region may be reconstructed based on the target part. As another example, an image may be reconstructed based on imaging data collected using a dopamine-related tracer, and a brain region may be marked in the image; or a target part corresponding to the brain region may be determined from the imaging data, and an image corresponding to the brain region may be reconstructed based on the target part.

In some embodiments, the determination module 220 or another computing device may generate the tracer classification model by training an initial model based on training data.

In some embodiments, the tracer classification model may be used in combination with other networks or models. For example, the tracer classification model may be applied jointly with an image reconstruction network, to directly generate the image based on the raw data through deep learning. Merely by way of example, the classification information of the tracer may be obtained by inputting the imaging data into the tracer classification model. Further, an image reconstruction model corresponding to the classification information may be obtained, and image reconstruction may be implemented by inputting the imaging data into the corresponding image reconstruction network. In some embodiments, the tracer classification model and the image reconstruction model may be jointly trained.

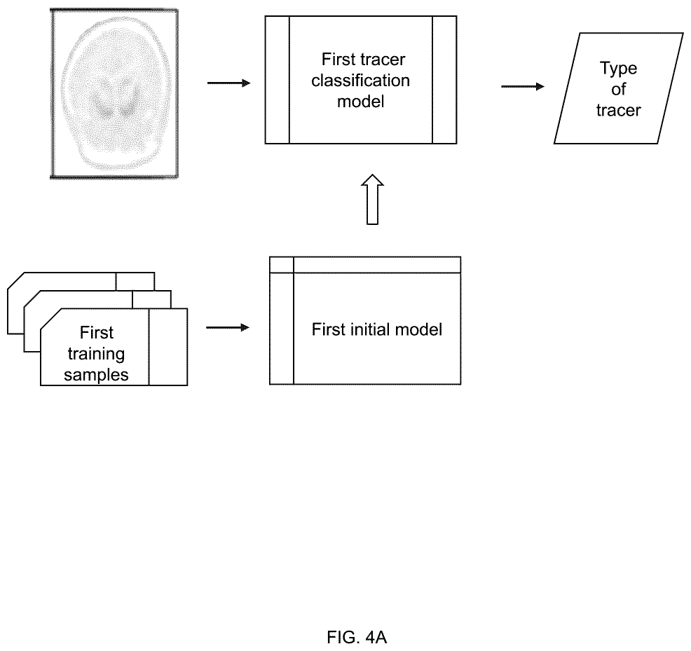

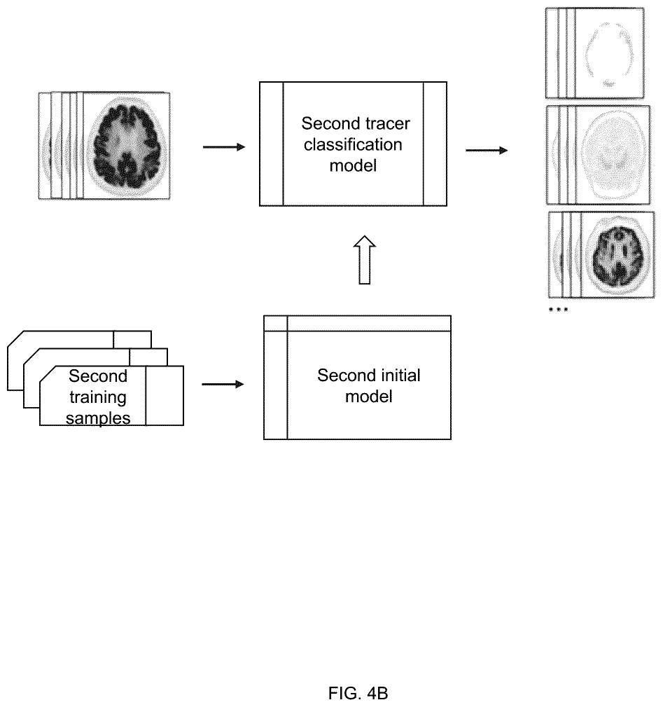

Merely by way of example, FIG. 4A is a schematic diagram illustrating a training and application process of the first tracer classification model according to some embodiments of the present disclosure. As shown in FIG. 4A, the determination module 220 may obtain the first tracer classification model by training a first initial model using first training samples. Each first training sample may include historical imaging data collected by a single scan and a type label of a tracer used in the scan, for example, a PET image collected by a single PET scan and a PET tracer label. During application, imaging data collected by a single ECT scan may be input into the first tracer classification model, and the first tracer classification model may output the type of the tracer used in the ECT scan.

FIG. 4B is a schematic diagram illustrating a training and application process of the second tracer classification model according to some embodiments of the present disclosure. As shown in FIG. 4B, the determination module 220 may obtain the second tracer classification model by training a second initial model using second training samples. Each second training sample may include a plurality of sets of historical imaging data and classification results of each set of historical imaging data. For example, each second training sample may include a plurality of PET images collected using the same tracer or different tracers, and the grouping result of the PET images based on the tracer types of the PET images. During application, imaging data collected by a plurality of ECT scans may be fed into the second tracer classification model, and the second tracer classification model may group the imaging data by the tracers used in the ECT scans (e.g., into a first set of imaging data collected using 18F-FDG, a second set of imaging data collected using 18F-FLT, etc.).

According to some embodiments of the present application, the second tracer classification model for processing imaging data collected in multiple scans and the first tracer classification model for processing imaging data collected in a single scan may be trained respectively. If imaging data collected in multiple scans and imaging data collected in a single scan both need to be processed, the first tracer classification model and the second tracer classification model may both be used, thereby improving the accuracy and efficiency of tracer classification.

As shown in FIG. 5, in some embodiments, the determination module 220 may determine classification information 540 of a tracer by processing imaging data 510 using a tracer classification model 520 and an enhancement model 530.

The enhancement model 530 may be a noise reduction model, which refers to a processing algorithm or model for filtering noise information contained in imaging data. In some embodiments, the enhancement model 530 may be a machine learning model, such as a UNET neural network model, a deep neural network, etc. In some embodiments, the enhancement model 530 may perform detail enhancement on the imaging data while performing noise reduction on the imaging data.

In some embodiments, the enhancement model 530 may include a universal enhancement model and/or an enhancement model corresponding to a specific type of tracer. For example, training samples of the universal enhancement network may include sample imaging data collected using a plurality of types of tracers, which may be used to perform noise reduction and/or detail enhancement on imaging data collected with various tracers. The noise reduction network for a specific type of tracer may be trained with sample imaging data collected using the specific type of tracer, and may be used to perform noise reduction and/or detail enhancement on imaging data collected using the specific type of tracer. In some embodiments, the detail enhancement may be performed to enhance image contrast, image brightness balance, etc. by performing histogram equalization, logarithmic transformation, Gamma transformation, etc. During the training of the noise reduction network, labels corresponding to the training samples may be obtained by performing detail enhancement on golden standard images corresponding to the training samples, thereby obtaining a noise reduction network having both a noise reduction function and a detail enhancement function, and such noise reduction network may be designated as the enhancement model.

In some embodiments, noise reduction of the imaging data 510 may be performed first based on the enhancement model 530 (e.g., the universal enhancement model), then the imaging data after noise reduction output by the enhancement model 530 may be input into the tracer classification model 520, and the tracer classification model 520 may output the classification information 540. Merely by way of example, a PET image collected by a certain PET scan may be first input into the enhancement model 530, and the enhancement model 530 may perform noise reduction (e.g., removing grain noise, Gaussian noise, etc.) and/or detail enhancement on the image; then the tracer classification model 520 may determine the classification information 540 by processing the PET image after noise reduction.

Noise reduction and/or detail enhancement may be performed on the imaging data based on the enhancement model 530 first, and then the classification information of the tracer corresponding to the imaging data after the processing may be determined by the tracer classification model 520, thereby reducing the impact of the noise data in the imaging data on tracer classification, and improving the accuracy of the classification result.

In some embodiments, the determination module 220 may first determine an initial classification of the tracer used in the imaging data based on the tracer classification model 520, and then obtain a specific enhancement model corresponding to the initial classification. Further, the determination module 220 may obtain the imaging data after noise reduction and/or detail enhancement by processing the imaging data using the corresponding enhancement model. The determination module 220 may obtain more accurate classification data by further processing the imaging data after noise reduction and/or detail enhancement based on the tracer classification model 520. For example, if the initial classification of the tracer is 18F-FMISO, the imaging data may be input into an enhancement model corresponding to 18F-FMISO for noise reduction. The tracer classification model 520 may further determine a final classification of the tracer based on the imaging data after noise reduction and/or detail enhancement. As imaging data collected with different tracers has different noise levels and is applicable to different enhancement models (e.g., different noise reduction networks corresponding to different noise levels), in some embodiments of the present disclosure, the enhancement model corresponding to the imaging data may be determined based on the initial classification. In this way, the effectiveness of noise reduction and the accuracy of the imaging data after noise reduction can be improved, thereby improving the accuracy of subsequent tracer classification.

In some embodiments, the tracer classification model 520 and the enhancement model 530 may be two separate models. Alternatively, the tracer classification model 520 and the enhancement model 530 may be connected with each other to form a cascaded model. For example, an output layer of the enhancement model 530 may be connected with an input layer of the tracer classification model 520. In some embodiments, the tracer classification model 520 and the enhancement model 530 may be trained and generated separately. More descriptions about the training method and the training data of the tracer classification model 520 may be found in FIG. 4A and FIG. 4B, which is not repeated here.

In some embodiments, the determination module 220 or another computing device may generate the enhancement model 530 by training an initial enhancement model using labeled historical imaging data. The label corresponding to historical imaging data may be historical imaging data after noise reduction. In some embodiments, the historical imaging data after noise reduction and/or detail enhancement may be manually processed or validated. For example, the determination module 220 may process the historical imaging data using an image noise reduction algorithm and/or a detail enhancement algorithm, and send the historical imaging data after noise reduction and/or detail enhancement to a user for confirmation, and use the historical imaging data after noise reduction and/or detail enhancement as a training label after user confirmation.

In some embodiments, the historical imaging data may be collected with different tracers, and the universal enhancement model described above may be trained based on the historical imaging data.

In some embodiments, the historical imaging data may be collected with a specific tracer, and the above-mentioned enhancement model corresponding to the specific tracer may be trained based on the historical imaging data. For example, if the historical imaging data is collected with a tracer 11C-MET, the trained enhancement model may perform noise reduction processing and/or detail enhancement processing on imaging data collected with the tracer 11C-MET. In some embodiments, the determination module 220 or another computing device may previously generate a plurality of enhancement models for a plurality of tracers for selection during tracer classification.

In some embodiments, the tracer classification model and the enhancement model may be generated by joint training. For example, parameters of the tracer classification model and the enhancement model may be obtained by training an initial processing model. The initial processing model may include a first initial component and a second initial component connected with each other. The first initial component may be used to perform noise reduction processing and/or detail enhancement processing on training data input into the initial processing model, and the second initial component may be used to determine classification information of a tracer used in collecting the training data. The output of the first initial component may be used as the input of the second initial component. In some embodiments, the training data of the initial processing model may include historical imaging data, historical imaging data after noise reduction and/or detail enhancement and classification information of a tracer used in collecting the historical imaging data. During training, the historical imaging data may be used as the input of the first initial component, and the output of the first initial component may include predicted imaging data after noise reduction and/or detail enhancement; the output of the first initial component may be used as the input of the second initial component, and the output of the second initial component may include predicted classification information of a tracer. After model training, the trained first initial component may be used as the enhancement model, and the trained second initial component may be used as the tracer classification model.

By determining parameters of the tracer classification model and the enhancement model through the above training methods, the utilization rate of the training data may be improved in some cases. For example, the obtained historical imaging data may be applied to train both the tracer classification model and the enhancement model. Besides, the model training efficiency can also be improved, such as achieving the purpose of obtaining two models through a single training process.

In some embodiments, the determining the classification information of the tracer by processing the imaging data using the tracer classification model and the enhancement model includes one or more iterations (i.e., is an iterative process). For illustration purposes, FIG. 6 is a schematic diagram illustrating a current iteration according to some embodiments of the present disclosure. In some embodiments, S1-S5 in FIG. 6 may all be performed by the determination module 220.

In S1, initial classification information of the tracer may be determined by processing initial imaging data of the iteration using the tracer classification model.

In the first iteration, the imaging data obtained in operation 310 may be used as the initial imaging data. In subsequent iterations such as the second iteration, the initial imaging data may be updated imaging data output from the previous iteration. In some embodiments, the imaging data may include imaging data collected in a single scan or an imaging data set collected in a plurality of scans as described above. If the initial imaging data is generated based on the imaging data set collected in a plurality of scans, the initial classification information may include classification information of a tracer used in each scan. If the initial imaging data is generated based on imaging data collected in a single scan, the initial classification information may include classification information of a tracer used in the scan. The process of determining the initial classification information of the tracer using the tracer classification model may be similar to the process of determining the classification information of the tracer using the tracer classification model as described in connection with operation 320 in FIG. 3, which is not repeated here.

In S2, updated imaging data of the iteration may be determined by processing the initial imaging data of the iteration using the enhancement model.

The updated imaging data may refer to imaging data obtained after performing noise reduction processing and/or detail enhancement processing on the initial imaging data using the enhancement model.

In some embodiments, the determination module 220 may obtain at least two candidate enhancement models corresponding to at least two tracer types; then select the enhancement model from the at least two candidate enhancement models based on the initial classification information; and generate the updated imaging data by performing noise reduction processing and/or detail enhancement processing on the initial imaging data using the enhancement model. For example, the enhancement model may include an enhancement model A corresponding to a tracer A, an enhancement model B corresponding to a tracer B, . . . , and an enhancement model N corresponding to a tracer N. If the tracer classification model determines that the tracer corresponding to the initial imaging data is the tracer A, the determination module 220 may obtain the updated imaging data after noise reduction and/or detail enhancement by performing noise reduction processing and/or detail enhancement processing on the initial imaging data using the enhancement model A.

In some embodiments, the initial imaging data may be a data set or an image set generated based on the imaging data set collected by a plurality of scans. The tracer classification model may group the initial imaging data into initial imaging data sets corresponding to different tracers. The determination module 220 may select the corresponding enhancement model for each initial imaging data set, and process the each initial imaging data set using the corresponding enhancement model. For example, the initial imaging data may be divided into an initial imaging data set corresponding to the tracer A, an initial imaging data set corresponding to the tracer B, etc. The determination module 220 may input the imaging data set of the tracer A into the enhancement model A for noise reduction and/or detail enhancement and input the imaging data set of the tracer B into the enhancement model B for noise reduction and/or detail enhancement.

In some embodiments, the candidate enhancement models may include candidate enhancement models for processing different types of imaging data. For example, the candidate enhancement models may include a first candidate enhancement model for processing raw data and a second candidate enhancement model for processing image data. The determination module 220 may select the first candidate enhancement model or the second candidate enhancement model as the enhancement model based on the type of the initial imaging data.

In S3, whether an iteration termination condition is satisfied may be determined.

In some embodiments, the iteration termination condition may include that iteration convergence is achieved, for example, the initial classification information obtained in two consecutive iterations is the same. In some embodiments, the iteration termination condition may include that a preset number of iterations have been performed, such as 10 iterations. The iteration termination condition may be manually set in advance or determined by the determination module 220 based on actual needs.

In response to a determination result that the iteration termination condition is not satisfied, the updated imaging data may be designated as initial imaging data of a next iteration by performing S4. In some embodiments, if the iteration termination condition is not satisfied, it may represent that the iterative process needs to be continued until the iteration termination condition is satisfied in a certain iteration.

In response to a determination result that the iteration termination condition is satisfied, the initial classification information may be designated as the classification information of the tracer by performing S5.

In some embodiments, in response to the determination result that the iteration termination condition is satisfied, the currently obtained initial classification information may be designed as final classification information of the tracer and the iterative process may be stopped. In some embodiments, in response to the determination result that the iteration termination condition is satisfied, the determination module 220 may determine the updated imaging data in the current iteration as the imaging data after noise reduction.

Determining the classification information of the tracer based on the iterative process can effectively improve the accuracy of classification information. In each iteration, the enhancement model corresponding to the initial imaging data may be selected based on the initial classification information, then noise reduction and/or detail enhancement may be performed on the initial imaging data based on the enhancement model, and the updated imaging data after noise reduction and/or detail enhancement may be input into the tracer classification model as the initial imaging data of the next iteration. That is to say, in other iterations except for the first iteration, the initial imaging data processed by the tracer classification model may be data after noise reduction and/or detail enhancement, which may improve the accuracy of tracer classification. In addition, in each iteration, compared with the initial imaging data of the previous iteration, the initial imaging data of the current iteration may have less noise data and better data quality, and the classification information obtained in each iteration may be more accurate compared with that of the previous iteration.

In some embodiments, the imaging data may include raw data and image data obtained in one or more scans of the target object, which may both be used to determine the classification information of the tracer. In different scan situations, the accuracy of the tracer classification result based on the raw data and the accuracy of the tracer classification result based on the image data may be different. In order to improve the accuracy of the final classification information, quality analysis may be performed on the raw data and/or the image data.

Merely by way of example, FIG. 7 is a schematic flowchart illustrating a process 700 for determining classification information of a tracer based on raw data and image data according to some embodiments of the present disclosure.

As shown in FIG. 7, the process 700 may include the following operations.

In 710, first classification information of the tracer may be determined by processing the raw data using a third tracer classification model.

The third tracer classification model may also be referred to as a tracer classification model corresponding to the raw data, which may determine the classification information of the tracer based on the input raw data. Training samples of the third tracer classification model may include historical raw data collected by single or a plurality of scans and a type label of the tracer used in each scan.

In some embodiments, the first classification information may include several raw data sets. Each raw data set may correspond to a specific type of tracer. For example, the raw data corresponding to a plurality of ECT scans of one or more target objects may be input into the third tracer classification model, and the third tracer classification model may output groups of raw data corresponding to different tracer types. In some embodiments, the first classification information may include classification information of the tracer used in a single ECT scan.

In 720, second classification information of the tracer may be determined by processing the image data using a fourth tracer classification model.

The fourth tracer classification model may also be referred to as a tracer classification model corresponding to the image data, which may determine the classification information of the tracer based on the input image data. Training samples of the fourth tracer classification model may include historical image data collected by a single or a plurality of scans and a type label of the tracer used in each scan.

In some embodiments, similar to the first classification information, the second classification information may include several image data sets or classification information of the tracer used in a single ECT scan. Each image set may correspond to a specific type of tracer.

In 730, a first weight of the first classification information and a second weight of the second classification information may be determined by performing a quality assessment on the image data.

In some embodiments, the determination module 220 may determine the classification information of the tracer based on the first classification information, the second classification information and the corresponding weight values thereof. For the convenience of description, the first classification information and the second classification information may be referred to as sub-classification information. A weight of the sub-classification information may reflect an importance of the sub-classification information and its influence on the finally determined classification information. For example, the greater the weight corresponding to the sub-classification information is, the more important the sub-classification information may be, and the greater its influence on the finally determined classification information may be.

In some embodiments, the first weight and the second weight may be determined by performing quality assessment on the image data. A quality assessment result of the image data may be represented by a quality score of the image data. For example, a sum of the first weight and the second weight may be 1. The higher the quality score of the image data is, the smaller the value of the first weight may be, and the larger the value of the second weight may be. That is to say, if the quality of the image data is good, the second classification information obtained based on the image data may have a greater impact on the final classification result than the first classification information obtained based on the raw data.

The quality assessment of the image data may be performed in various ways. For example, the determination module 220 may determine the quality score of the image data based on a noise level of the image data and/or a data volume of the image data. The higher the noise level of the image data is and/or the smaller the data volume of the image data is, the lower the quality score of the image data may be. In some embodiments, the data volume of the image data may refer to a data volume of the raw data corresponding to the image data (i.e., a reconstruction basis of the image data). Generally, the image data may have a higher noise level and a poorer quality if it is reconstructed from raw data with a smaller data volume.

In some embodiments, the determination module 220 may determine the quality score of the image data based on a processing result of the image data by a quality assessment model, the quality assessment model being a trained machine learning model. For example, the quality evaluation model may include a deep neural network (DNN) model, a convolutional neural network (CNN) model, a recurrent neural network (RNN) model, a graph neural network (GNN) model, etc. or any combination thereof.

In some embodiments, an input of the quality assessment model may include the image data, and an output of the quality assessment model may be the quality score of the image data. For example, the quality score of the image data may be represented by a number between 0-1, and the larger the number is, the higher the quality score may be. In some embodiments, the quality assessment model may be generated by training an initial quality assessment model with labeled training samples. Each training sample may include sample image data, and the label of each training sample may include a score of the corresponding sample image data. The score of the sample image data may be determined by a user or by the determination module 220.

In 740, the classification information of the tracer may be determined based on the first classification information, the second classification information, the first weight, and the second weight.

In some embodiments, the classification information of the tracer may be a weighted sum of the sub-classification information and corresponding weight values thereof.

In some embodiments, the classification information of the tracer may be classification information corresponding to the sub-classification information whose weight value satisfies a preset condition. For example, the preset condition may be that the weight value is greater than or equal to a preset threshold, such as 0.8. If the weight value of the first weight corresponding to the first classification information is 0.9, and the weight value of the second weight corresponding to the second classification information is 0.1, the determination module 220 may determine the first classification information as the classification information of the tracer.

The above description about determining the classification information of the tracer is only an example, and the determination module 220 may determine the classification information of the tracer by flexibly applying the above method. For example, the determination module 220 may determine the quality score of the image data by performing quality assessment on the image data. If the quality score of the image data is greater than a first score threshold (e.g., 0.8), the determination module 220 may determine the classification information for the tracer by directly processing the image data using the fourth tracer classification model. As another example, if the quality score of the image data is less than a second score threshold (e.g., 0.2), the determination module 220 may determine the classification information of the tracer by directly processing the raw data using the third tracer classification model. As another example, the aforementioned operations 710 and 720 may be performed in any order or in parallel.

As the image data and the raw data contain different information, determining the classification information of tracers based on both the image data and the raw data may improve the accuracy of tracer classification. Moreover, the weight corresponding to each sub-classification information is determined based on the quality of the image data, and then the final classification information may be determined based on the weight of each sub-classification, thereby further improving the accuracy of the determined classification information.

In some embodiments, the determination module 220 may first predict the classification information of the tracer based on some reference information (e.g., a disease type, a disease, etc., of the target object), and obtain the initial classification information of the tracer, and then determine the classification information of the tracer by processing the imaging data and the initial classification information using the tracer classification model.

Merely by way of example, FIG. 8 is a schematic flowchart illustrating a process 800 for determining classification information of a tracer based on imaging data and reference information. As shown in FIG. 8, the process 800 may include the following operations.

In 810, reference information may be obtained.

The reference information may be used as a reference basis for determining the classification of the tracer. In some embodiments, the reference information may include one or more historical diagnosis reports of the target object, doctor's annotations (e.g., marked points and/or regions) on one or more historical scan images of the target object, a target region of the scan, a lesion region of the target object, a disease type and symptoms of the target object, etc.

In some embodiments, the reference information may be obtained by the determination module 220 based on historical diagnosis and treatment information of the target object. In some embodiments, the reference information may be inputted by a user and/or obtained from a storage device.

In 820, the initial classification information of the tracer may be based on the reference information.

In some embodiments, the determination module 220 may determine the initial classification information of the tracer based on the obtained reference information. Normally, the tracer used in the scan is selected according to a type of a region of interest (ROI) of the target object. Usually, a specific ROI may be imaged with a specific tracer. Therefore, the determination module 220 may infer the type of the tracer used in the scan based on the ROI of the target object, as the initial classification information of the tracer. The ROI of the target object may include one or more points and/or regions marked by a doctor in one or more historical scan images of the target object described in operation 810, the target region of the scan, the lesion region of the target object, etc. As another example, the tracer used in the scan is normally selected according to a disease type and/or symptoms of the target object. Usually, a specific disease type and/or symptoms may be imaged with a specific tracer. Therefore, the determination module 220 may infer the type of the tracer used in the scan based on the disease type and/or symptoms of the target object, as the initial classification information of the tracer. Merely by way of example, if the target object has prostate cancer, it may be inferred that the tracer used in the scan is PSMA.

In some embodiments, the initial classification information of the tracer may include initial probabilities of various tracer types, wherein an initial probability of each tracer type indicates a probability that the tracer used in the current scan belongs to the tracer type. The determination module 220 may determine the initial probability of each tracer type based on the reference information. If a first tracer type has a higher initial probability than a second tracer type, the tracer of the first tracer type may have a greater probability of being used during the current scan. Merely by way of example, it is assumed that the reference information includes the target region of the scan. If the target region of the scan is the prostate of the target object, the determination module 220 may infer that the tracer used in the scan is PSMA, then the determination module 220 may set a higher initial probability for a PSMA-related tracer. Optionally, the initial classification information of the tracer may be represented by a vector including the initial probabilities of various tracer types. Each vector element of the vector may correspond to the initial probability of one type of tracer.

In 830, the classification information of the tracer may be determined by processing the imaging data and the initial classification information using the tracer classification model.

In some embodiments, the classification information of the tracer may be obtained by inputting the initial classification information (e.g., in the form of a vector) and the imaging data into the tracer classification model. It should be understood that during training, training samples of the tracer classification model may also include sample initial classification information, which may be determined based on sample reference information corresponding to the training samples.

The accuracy and efficiency of the tracer classification model can be improved by determining the classification information of the tracer based on the initial classification information determined based on the reference information. In some embodiments, 820 may be omitted, and the classification information of the tracer may be obtained by directly inputting the reference information and the imaging data into the tracer classification model.

In some embodiments, in addition to being used to determine the initial classification information of the tracer, the reference information may also be used to verify the accuracy of the classification information of the tracer determined by the tracer classification model. For example, after operation 320 or 830, the determination module 220 may determine whether the difference between the classification information and the initial classification information of the tracer satisfies a preset condition, and reconfirm the classification information of the tracer in response to the determination result that the preset condition is satisfied.

In some embodiments, the preset condition may be that the classification information of the tracer is different from the initial classification information or that a degree of the difference is relatively large. For example, the preset condition may be that tracer types corresponding to the classification information and the initial classification information belong to different radionuclides or organic ligands. As another example, the classification information includes a first probability of belonging to a specific tracer type, the initial classification information includes a second probability of belonging to the specific tracer type, and the preset condition may be that a difference between the first and second probabilities is greater than a difference threshold.

In some embodiments, if the preset condition is satisfied, the imaging data, the initial classification information, and the classification information may be sent to a user for manual confirmation and/or modification. In some embodiments, the determination module 220 may re-perform the method for classifying the tracer disclosed herein to reconfirm the classification information of the tracer.

The initial classification information may be determined based on the reference information, and the initial classification information may be used as a reference for evaluating the classification information output by the model, so that the classification information can be reviewed and adjusted in time in case of abnormality occurs in data processing, and the accuracy of the determined classification information of the tracer can be improved.

As mentioned above, the ROI type of the target object may be related to the type of tracer used. Usually, a specific ROI may be imaged with a specific tracer. Therefore, information related to the ROI may also be used as model input to assist in the determination of the classification information of the tracer. In some embodiments, the determination module 220 may determine the ROI based on the imaging data, generate a feature map representing the ROI, and then determine the classification information of the tracer by processing the imaging data and the feature map using the tracer classification model.

Merely by way of example, FIG. 9 is a schematic flowchart illustrating a process 900 for determining classification information of a tracer based on imaging data and a feature map according to some embodiments of the present disclosure. As shown in FIG. 9, the process 900 may include the following operations.

In 910, the ROI of the target object may be determined based on the imaging data.

In some embodiments, the imaging data may include image data. Alternatively, the imaging data may include raw data. The image data may be generated by reconstructing the raw data. The determination module 220 may send the image data to a user terminal, and determine the ROI based on an annotation input by the user. Alternatively, the determination module 220 may determine the ROI by analyzing the image data. For example, a lesion region may be segmented from the image data as the ROI by performing lesion detection.

In some embodiments, the determination module 220 may determine a region in the image data where a tracer uptake value (e.g., standard uptake value) is greater than an uptake threshold as the ROI. The uptake threshold may be a preset tracer uptake value for distinguishing the ROI from other regions. In general, a region with a higher uptake value may be more likely to be a lesion region (i.e. ROI). Determining the ROI based on the uptake threshold may improve the processing efficiency of the image data, and make the subsequent analysis more targeted, thereby making the determined classification result of the tracer more accurate.

In 920, a feature map representing the ROI may be determined based on the ROI.

The feature map may reflect a distribution of the ROI in the target object. In some embodiments, the feature map may have the same image size as the image data. The difference between the feature map and the image data may be that the ROI and the other regions in the feature map are displayed in different manners. For example, in the feature map, pixels belonging to the ROI may have larger pixel values (e.g., each has a pixel value of 1), and pixels belonging to other regions may have smaller pixel values (e.g., each has a pixel value of 0). If there is a plurality of ROIs, different pixel values may be set for different ROIs.

In 930, the classification information of the tracer may be determined by processing the imaging data and the feature map using the tracer classification model.

For example, the image data and the feature map may be input into the tracer classification model, and the tracer classification model may output the classification information. Optionally, the image data and the feature map may be input into the tracer classification model after being concatenated. It should be understood that if the input of the tracer classification model includes the feature map, each training sample of the tracer classification model may also include a sample feature map. The tracer classification model may focus on the analysis of the ROI when determining the classification information of the tracer based on the feature map of the region of interest, thereby reducing the amount of calculation and improving the processing efficiency and accuracy of the model.

In some embodiments, the imaging data may include an ECT image (e.g., a PET image, and a SPECT image) generated after reconstruction of the raw data. As the ECT image contains less and fuzzy structural information, the determination module 220 may generate a reference image containing more structural information based on the imaging data, and obtain more accurate classification information of the tracer by using the reference image as the input of the tracer classification model.

Merely by way of example, FIG. 10 is a schematic flowchart illustrating a process 1000 for determining classification information of a tracer based on a reference image according to some embodiments of the present disclosure. As shown in FIG. 10, the process 1000 may include the following operations.

In 1010, a pseudo MR image of the target object may be generated based on the imaging data.

The pseudo MR image may refer to a simulated MR image generated by the determination module 220 based on the imaging data, which may have a higher definition and more structural information than the ECT image.

In some embodiments, the determination module 220 may convert the ECT image into the corresponding pseudo MR image using a trained image conversion model. The image conversion model may be a trained machine learning model. For example, the image conversion model may include a deep neural network (DNN) model, a convolutional neural network (CNN) model, a recurrent neural network (RNN) model, a graph neural networks (GNN) model, etc. or any combination thereof. In some embodiments, an input of the image conversion model may include the ECT image, and an output of the image conversion model may include the pseudo MR image.

In some embodiments, the image conversion model may be trained and generated using labeled training samples. A training sample may include a sample ECT image of a sample object, and the training label of the training sample may include a sample MR image of the corresponding to sample object. For example, the sample ECT image may be obtained by performing an ECT scan on the sample object, and the corresponding sample MR image may be obtained by performing MR scan on the sample object.

In 1020, the classification information of the tracer may be determined by processing the pseudo MR image using the tracer classification model.

For example, the pseudo MR image may be input into the tracer classification model, and the tracer classification model may output the classification information. As another example, the ECT image and the pseudo MR image may be input together into the tracer classification model, and the tracer classification model may output the classification information. Optionally, the ECT image and the pseudo MR image may be input into the tracer classification model after being concatenated. It should be understood that if the input of the tracer classification model includes the pseudo MR image, each training sample of the tracer classification model may also include a sample pseudo MR image.

The pseudo MR image including more structural information may be input into the tracer classification model for processing, thereby providing additional reference information for the tracer classification and improving the accuracy of the model output.

It should be noted that the above descriptions of the process 800, 900, and 1000 are for illustrative purposes only, and do not limit the scope of the application of the present disclosure. Those skilled in the art may make various modifications and alterations to the process 800, 900, and 1000 under the teachings of the present disclosure. However, such modifications and alterations are still within the scope of the present disclosure. For example, the feature map may be generated by performing operations 910 and 920, the pseudo MR image may be generated by performing operation 1010, and the classification information of the tracer may be determined by using the imaging data, the feature map, and the pseudo MR image together as the input of the tracer classification model. As another example, a pseudo CT image, instead of the pseudo MR image, may be generated and used in determining the classification information.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, the embodiments of the present disclosure are not limited to that precisely as shown and described.

What is claimed is:

1. A method for classifying a tracer, comprising:
obtaining imaging data related to an emission computed tomography (ECT) scan of a target object, the target object being injected with a tracer during the ECT scan; and
determining classification information of the tracer by processing the imaging data using a tracer classification model, the tracer classification model being a trained machine learning model.

2. The method of claim 1, wherein the determining classification information of the tracer by processing the imaging data using a tracer classification model includes:
determining at least one feature of the imaging data; and
determining the classification information of the tracer by processing the imaging data and the at least one feature using the tracer classification model.

3. The method of claim 1, wherein the determining classification information of the tracer by processing the imaging data using a tracer classification model includes:
determining the classification information of the tracer by processing the imaging data using the tracer classification model and an enhancement model.

4. The method of claim 3, wherein the determining the classification information of the tracer by processing the imaging data using the tracer classification model and an enhancement model includes one or more iterations, an iteration of the one or more iterations including:
determining initial classification information of the tracer by processing initial imaging data of the iteration using the tracer classification model;
generating updated imaging data by performing noise reduction processing on the initial imaging data using the enhancement model;
determining whether an iteration termination condition is satisfied; and
designating the initial classification information as the classification information of the tracer in response to a determination result that the iteration termination condition is satisfied; or designating the updated imaging data as initial imaging data of a next iteration in response to a determination result that the iteration termination condition is not satisfied.

5. The method of claim 4, wherein the generating updated imaging data by performing noise reduction processing on the initial imaging data using the enhancement model includes:
obtaining at least two candidate enhancement models corresponding to at least two tracer types;
selecting the enhancement model from the at least two candidate enhancement models based on the initial classification information; and
generating the updated imaging data by performing the noise reduction processing on the initial imaging data using the enhancement model.

6. The method of claim 3, wherein the tracer classification model and the enhancement model are generated by joint training.

7. The method of claim 1, wherein the imaging data includes raw data and image data collected based on the ECT scan of the target object;
the determining classification information of the tracer by processing the imaging data using a tracer classification model includes:
determining first classification information of the tracer by processing the raw data using a tracer classification model corresponding to the raw data;
determining second classification information of the tracer by processing the image data using the tracer classification model corresponding to the image data;
determining a first weight of the first classification information and a second weight of the second classification information by performing quality assessment on the image data; and
determining the classification information of the tracer based on the first classification information, the second classification information, the first weight, and the second weight.

8. The method of claim 1, wherein the determining classification information of the tracer by processing the imaging data using a tracer classification model includes:
obtaining reference information;
determining initial classification information of the tracer based on the reference information; and
determining the classification information of the tracer by processing the imaging data and the initial classification information using the tracer classification model.

9. The method of claim 1, wherein the determining classification information of the tracer by processing the imaging data using a tracer classification model includes:
determining a region of interest based on the imaging data;
determining a feature map representing the region of interest; and
determining the classification information of the tracer by processing the imaging data and the feature map using the tracer classification model.

10. The method of claim 1, the determining classification information of the tracer by processing the imaging data using a tracer classification model includes:
generating a pseudo magnetic resonance (MR) image of the target object based on the imaging data; and
determining the classification information of the tracer by processing the pseudo MR image using the tracer classification model.

11. A system, comprising:
at least one storage device storing a set of instructions for classifying a tracer; and
at least one processor configured to communicate with the at least one storage device, wherein when executing the set of instructions, the at least one processor is configured to direct the system to perform operations including:
obtaining imaging data related to an emission computed tomography (ECT) scan of a target object, the target object being injected with a tracer during the ECT scan; and
determining classification information of the tracer by processing the imaging data using a tracer classification model, the tracer classification model being a trained machine learning model.

12. The system of claim 11, wherein the determining classification information of the tracer by processing the imaging data using a tracer classification model includes:
determining at least one feature of the imaging data; and
determining the classification information of the tracer by processing the imaging data and the at least one feature using the tracer classification model.

13. The system of claim 11, wherein the determining classification information of the tracer by processing the imaging data using a tracer classification model includes:
determining the classification information of the tracer by processing the imaging data using the tracer classification model and an enhancement model.

14. The system of claim 13, wherein the determining the classification information of the tracer by processing the imaging data using the tracer classification model and an enhancement model includes one or more iterations, an iteration of the one or more iterations including:
determining initial classification information of the tracer by processing initial imaging data of the iteration using the tracer classification model;
generating updated imaging data by performing noise reduction processing on the initial imaging data using the enhancement model;
determining whether an iteration termination condition is satisfied; and
designating the initial classification information as the classification information of the tracer in response to a determination result that the iteration termination condition is satisfied; or designating the updated imaging data as initial imaging data of a next iteration in response to a determination result that the iteration termination condition is not satisfied.

15. The system of claim 14, wherein the generating updated imaging data by performing noise reduction processing on the initial imaging data using the enhancement model includes:
obtaining at least two candidate enhancement models corresponding to at least two tracer types;
selecting the enhancement model from the at least two candidate enhancement models based on the initial classification information; and
generating the updated imaging data by performing the noise reduction processing on the initial imaging data using the enhancement model.

16. The system of claim 13, wherein the tracer classification model and the enhancement model are generated by joint training.

17. The system of claim 11, wherein the imaging data includes raw data and image data collected based on the ECT scan of the target object;
the determining classification information of the tracer by processing the imaging data using a tracer classification model includes:
determining first classification information of the tracer by processing the raw data using a tracer classification model corresponding to the raw data;
determining second classification information of the tracer by processing the image data using the tracer classification model corresponding to the image data;
determining a first weight of the first classification information and a second weight of the second classification information by performing quality assessment on the image data; and
determining the classification information of the tracer based on the first classification information, the second classification information, the first weight, and the second weight.

18. The system of claim 11, wherein the determining classification information of the tracer by processing the imaging data using a tracer classification model includes:
obtaining reference information;
determining initial classification information of the tracer based on the reference information; and
determining the classification information of the tracer by processing the imaging data and the initial classification information using the tracer classification model.

19. The system of claim 1, wherein the determining classification information of the tracer by processing the imaging data using a tracer classification model includes:
determining a region of interest based on the imaging data;
determining a feature map representing the region of interest; and
determining the classification information of the tracer by processing the imaging data and the feature map using the tracer classification model.

20. A non-transitory computer readable medium, comprising a set of instructions for classifying a tracer, wherein when executed by at least one processor, the set of instructions direct the at least one processor to effectuate a method, the method comprising:
obtaining imaging data related to an emission computed tomography (ECT) scan of a target object, the target object being injected with a tracer during the ECT scan; and
determining classification information of the tracer by processing the imaging data using a tracer classification model, the tracer classification model being a trained machine learning model.
